(19) United States
(12) Patent Application Publication
(10) Pub. No.: US 2010/0242955 A1
(43) Pub. Date: Sep. 30, 2010

(54) Aerosolized Drug Delivery System

(76) Inventor: Leland G. Hansen, St. Paul, MN (US)

Correspondence Address: BRIGGS AND MORGAN P.A., 2200 IDS CENTER, 80 SOUTH 8TH ST, MINNEAPOLIS, MN, 55402, US

(21) Appl. No.: 12/728,189

(22) Filed: Mar. 19, 2010

Related U.S. Application Data
(60) Provisional application No. 61/161,582, filed on Mar. 19, 2009.

(51) Int. Cl. A61M 11/00; G06F 19/00

(52) U.S. Cl. 128/200.23; 702/19

(57) ABSTRACT

A system for delivering an aerosolized drug to a patient includes an aerosol drug generator coupled to a mouthpiece including two sensing ports. A pressure sensor is connected to the two sensing ports of the mouthpiece. The system also includes a data processing component which calculates an inspired flow rate based on a signal from the pressure sensor, and a measurement component which measures the inhalation time, which is the time during which the aerosolized drug is inhaled at the inspired flow rate. The data processing component also calculates the amount of the aerosolized drug delivered to the patient based on the inspired flow rate, the amount of inhalation time, and a drug delivery coefficient.

### RELATED APPLICATIONS

This application claims priority under 35 U.S.C. §119(e) to U.S. Provisional Patient Application No. 61/161,582, filed Mar. 19, 2009, the contents of which are incorporated herein by reference.

### TECHNICAL FIELD

The present disclosure is generally directed to aerosol drug delivery and more specifically to devices and methods for delivering pharmaceuticals to a patient during an aerosol therapy session.

### BACKGROUND OF THE INVENTION

Aerosol therapy has recognized clinical advantages over intravenous or oral drug therapy. The advantages include a higher therapeutic effect with a given dose of drug, fewer side effects, and more rapid action of the drug. See Stephen P. Newman & Stewart W. Clarke, Therapeutic Aerosols—Physical and Practical Considerations, 38 Thorax 881 (1983); Nils Svedmyr, Clinical Advantages of the Aerosol Route of Drug Administration, 36 Respiratory Care 922 (1991); and Stephen P. Newman, Aerosol Deposition Considerations in Inhalation Therapy, 88 Chest 152S (1985), each reference being incorporated herein by reference for all purposes.

Some drugs designed to treat airway dysfunction are more effective when delivered via an aerosol. See Sam P. Giordano, Aerosol therapy: The Hard Questions, 36 Respiratory Care 914 (1991); and J. Jendle et al., Delivery and Retention of an Insulin Aerosol Produced by a New Jet Nebulizer, 8 Journal of Aerosol Medicine 243 (1995), each reference being incorporated herein by reference for all purposes. It is predicted that in the future, aerosol therapy will become a primary mode of drug delivery to deliver drugs to cystic fibrosis patients, and to deliver insulin to patients with diabetes mellitus.

Quantization of aerosolized drug delivered to a patient has not previously been possible. Patients' inhalation flow rates vary, and high inhalation air flow rates result in less deposition in the smaller air ways. Inhalation flow rate is one factor that influences where the aerosol is deposited in the patient's airway. The general instruction to the patient is to inhale slowly so as to allow the aerosol to penetrate deep in the lungs. The patient has little idea as to what slow means or what is the appropriate inhalation flow rate. Patient pauses for talking, coughing or resting result in significant loss of aerosolized drug to the environment. Under these conditions determining the dosage a patient actually receives is at best a guess.

Typically less than fifty percent of drug administered as aerosol reaches the lungs of the patient. The remainder is lost to the environment or remains as droplets in the aerosol generating device. Concerns have been raised about the health risk to primary care givers exposed to the aerosol lost to the environment, and about the cost effectiveness of aerosol delivery systems. Large investments have been made in aerosol drug research but few resources have been allotted to applied research on more effective ways of administering aerosol therapy and monitoring delivery of the drugs to the patient.

### BRIEF SUMMARY OF THE INVENTION

The present invention is directed to a system for delivering an aerosolized drug to a patient. In one embodiment of the aerosolized drug delivery system of the present invention, the system includes an aerosol drug generator coupled to a mouthpiece including two sensing ports. A pressure sensor is connected to the two sensing ports of the mouthpiece. The system also includes a data processing component which calculates an inspired flow rate based on a signal from the pressure sensor, and a measurement component which measures the inhalation time, which is the time during which the aerosolized drug is inhaled at the inspired flow rate. The data processing component may also calculate an amount of the aerosolized drug delivered to the patient based on the inspired flow rate, the amount of inhalation time, and a drug delivery coefficient. The system may include a display component for displaying the results of the calculations of the data processing component.

In another embodiment of the present invention, the aerosolized drug delivery system includes an aerosol drug generator coupled to a mouthpiece including two sensing ports, a pressure sensor connected to the two sensing ports, and a signal receiving component which receives signals from the pressure sensor. The system also includes a data processing component which calculates a plurality of inspired flow rates based on the signals from the pressure sensor. The system may also include a display component comprising a plurality of indicator lights, wherein each of the indicator lights corresponds to at least one inspired flow rate.

The present invention is also directed to a method of estimating an amount of aerosolized drug delivered to a patient during a therapy session using an aerosol generator. In one embodiment of the present invention, this method includes calculating an inspired flow rate as the patient inhales an aerosolized drug through a mouthpiece, by sensing a pressure differential across the mouthpiece. The method also includes measuring the inhalation time, which is the time during which the aerosolized drug is inhaled at the inspired flow rate. The method further includes calculating the amount of aerosolized drug delivered to the patient based on the inspired flow rate, the amount of inhalation time, and a drug delivery coefficient.

Embodiments of the present invention provide an inexpensive system for calculating the total amount of aerosol drug delivered to a patient during an aerosol therapy session. The system is user friendly and provides the care giver with a more accurate idea as to how much aerosolized drug was actually delivered to the patient during an individual therapy session.

In some embodiments, the system acts as an inhalation breath trainer that visually shows the patient his or her inhalation air flow rates. This feature allows each patient to adjust his or her inhalation flow rate to maximize drug deposition.

In some embodiments, the system acts as a compliance monitor which monitors the percentage of total time during a therapy session that the patient spent inhaling, and at what flow rates. The system may also be used as an aerosol control device to deliver aerosol to the patient only during the inhalation portion of the breathing cycle.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a graph of data representing weight of aerosol collected versus L/sec air flow in an experiment conducted using an aerosolized drug delivery system of the present invention.

FIG. 2 is a graph of data representing actual weight of aerosol delivered versus calculated weight of aerosol delivered during a simulated therapy session using a PARI Model 85B0000 device as part of an aerosolized drug delivery system of the present invention.

FIG. 3 is a graph of data representing actual weight of aerosol delivered versus calculated weight of aerosol delivered during a simulated therapy session using a PARI Trek® S nebulizer as part of an aerosolized drug delivery system of the present invention.

FIG. 4 depicts an aerosolized drug delivery system of the present invention.

FIG. 5 is a perspective view of a mouthpiece used in the aerosolized drug delivery system of the present invention.

FIG. 6 is a top view of the mouthpiece of FIG. 5.

FIG. 7 is a side view of the mouthpiece of FIG. 5.

FIG. 8 is a cross sectional view of the mouthpiece taken along line 5-5 in FIG. 6.

FIG. 9 is a functional schematic of the system of FIG. 4.

### DETAILED DESCRIPTION OF THE INVENTION

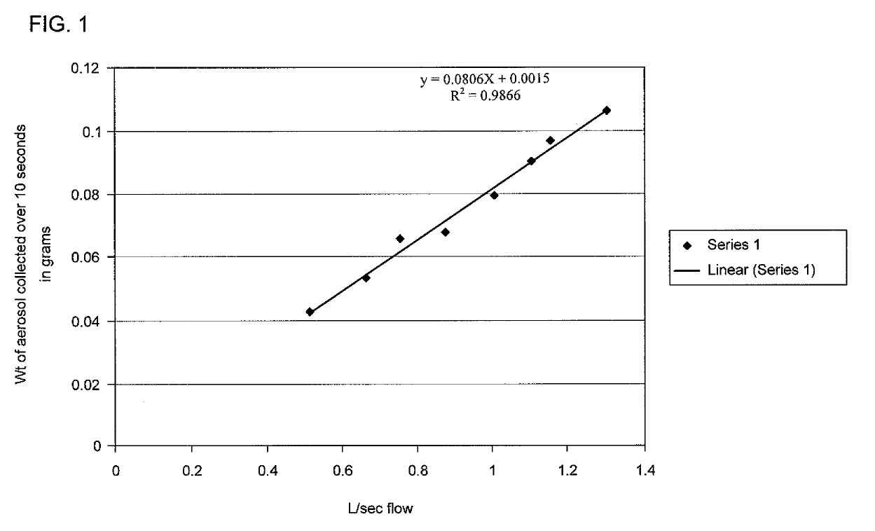

An aerosolized drug delivery system in accordance with the present invention functions as an aerosol drug delivery estimator. The amount of aerosol delivered to a patient at different air flow rates has been quantified experimentally. Higher inhalation air flow rates deliver more aerosol, from a constant flow aerosol generator, per unit of time. This is shown in FIG. 1, which is a graph of the weight of aerosol collected over 10 seconds versus air flow rate. The system of the present invention calculates the amount of aerosol delivered to the patient's mouth, based on the time spent at measured inhalation air flow rates, or “inspired flow rates,” during a therapy session. This system can be calibrated to any aerosol generating device. In accordance with this system, a microprocessor based data collector reads the pressure differential on two sides of a venturi-effect opening of a mouthpiece. The air flow rate data is calculated from the pressure data. The variable air flow rate data is integrated and total aerosol delivered to patient during that therapy session is calculated.

An aerosolized drug delivery system in accordance with the present invention may also function as a compliance monitor. When functioning as a compliance monitor, the system measures the amount of time in a therapy session that a patient was inhaling, and calculates the total percentage of time during the therapy session in which the patient was inhaling. The system also breaks down the percentage of time that the patient was inhaling at various inspired flow rates. The data obtained through the use of the system provides the care-giver or patient with information as to where the bulk of the aerosol from a given therapy session was deposited in the lungs, based on the inhalation flow rates. The data also allows the care-giver or patient to monitor how much time the patient spent inhaling the aerosol during a therapy session.

An aerosolized drug delivery system in accordance with the present invention may provide a continuous visual indicator of inhalation air flow rates. This allows the patient to visualize his or her inspired flow rate during the therapy session. Specifically, the inspired flow rate may be measured continuously and displayed on a display screen or monitor, or plotted on a streaming electronic air flow indicator graph. The continuous visual indicator of inspired flow rates is a training feature which allows the patient to adjust his or her inhalation air flow rate to a desired level during the therapy session.

An aerosolized drug delivery system of the present invention may also function as an aerosol controller. When functioning as an aerosol controller, the system provides the ability to control a two way air flow valve. This valve may control the air flow through the aerosol drug generator during patient expiration. The valve may be activated in response to a predetermined pressure signal. This feature allows the compressed air stream of a pneumatic nebulizer to be diverted temporarily, thus preventing aerosol production when the patient is not inhaling. This same feature can be used to control the output of other types of aerosol generators as well.

Embodiments of the present invention include a system of capturing and measuring the weight of water vapor from a constant output aerosol generator air stream. By using this system, the weight of aerosol delivered over a given period of time at a particular air flow rate can be determined. Since each air flow rate can carry a different amount of aerosolized drug, a different drug delivery coefficient for each air flow rate is used to calculate the amount of aerosolized drug delivered at a given inspired flow rate. See FIG. 1. Drug delivery coefficients may be expressed as the amount (in weight) of aerosol delivered in a given amount of time at a given inspired flow rate. The use of drug delivery coefficients allows the amount of drug delivered to a patient to be calculated, based on the measured inspired air flows and the amount of time spent at each measured inspired air flow. The aerosol delivered in the experiment which generated the data plotted in FIG. 1 was based on the constant output of the PARI Pro Nebulizer. In this experiment, the relationship between aerosol delivered to a patient, measured as captured aerosol water vapor, and the air flow rate over ten seconds showed a strong linear correlation, with an R² value of 0.9866. Other brands of nebulizers may require initial calibration of the device to ensure accurate results.

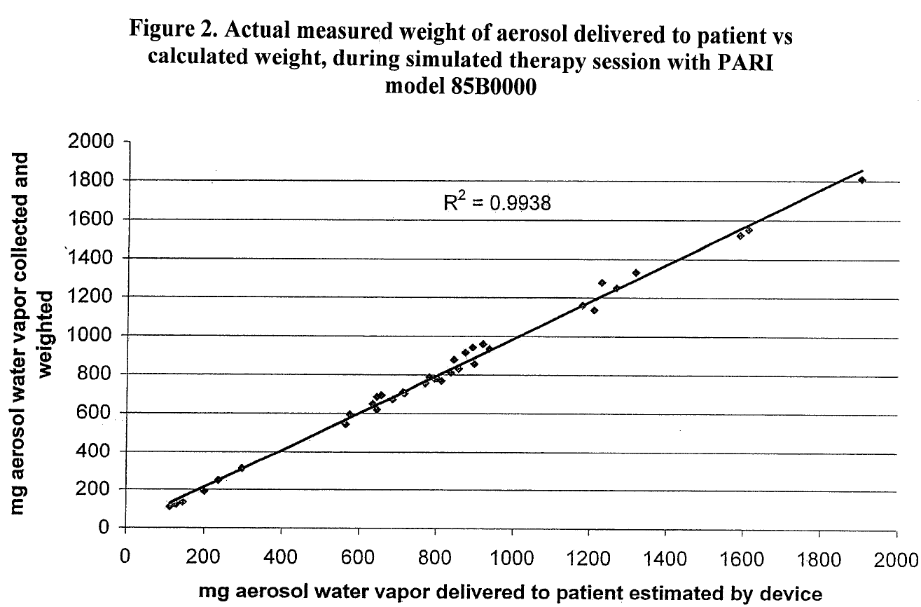

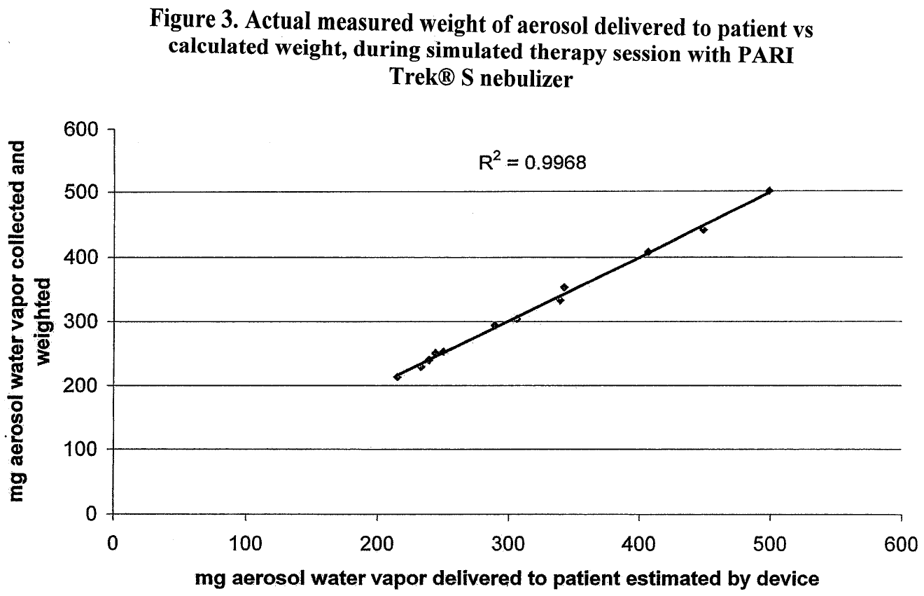

A device for measuring aerosol water/drug vapor delivered to a patient during aerosol therapy has been developed. Multiple comparisons under aerosol therapy conditions were conducted using the delivery estimator device of the present invention, and the water vapor trap system. The weight of the trapped water vapor was compared to the calculated estimates and plotted in FIGS. 2 and 3 for two models of PARI brand nebulizers. A very strong linear correlation between the estimated value and the actual trapped water vapor was observed, with the plots of FIGS. 2 and 3 having R² values of 0.9965 and 0.9968 respectively. Using the aerosol water vapor trap system as the standard, the estimated results were all within 6 percent of the actual measured water vapor amount for 37 different tests. The average deviation from the actual measured water vapor amount was 0.54 percent. (See FIG. 2.)

The LED bar graph trainer feature of embodiments of the present invention, which shows air flow rates, responded well to changes in air flow rate of a simulated breath cycle and compared accurately to the flow rates measured using a calibrated MANOSTAT flow meter calibrated at 20° C., with an accuracy of 2%.

Total percent time measured in the inhalation mode of the test period was checked with a stop watch and found to be accurate. The breakdown of the total inhalation percentage of time into percentage of time spent in each of three flow rate components proved to be difficult to measure with a stop watch in a simulated breathing test. When the air flow rate was held at any of three flow rates and timed, it closely matched the measured times.

The system of the present invention may utilize a mouth piece with a sensing port located on either side of a venturi opening. A single ultra-sensitive, dual port, amplified, negative pressure sensor, operating in the differential pressure mode, may be connected to these ports. The amplified signal from the negative pressure sensor is routed to a microprocessor based data collector that reads the pressure differential on each side of the venturi opening of the mouthpiece. This allows for continuous time based analysis of inspired air flow rate over the entire aerosol therapy session. The differential pressure may be sampled at various rates. For example, the pressure may be sampled at a rate of 16 samples per second. Each sample is used to calculate the air flow rate and time spent at that flow rate. The data may be accumulated in one of a series of storage areas, and each storage area may correspond to a different air flow rate. The amount of aerosolized drug is calculated based on the measured amount of time that air flows through the mouthpiece at each inspired flow rate. Each flow rate carries a different amount of aerosolized drug; therefore, a different drug delivery coefficient is used for each of the flow rates.

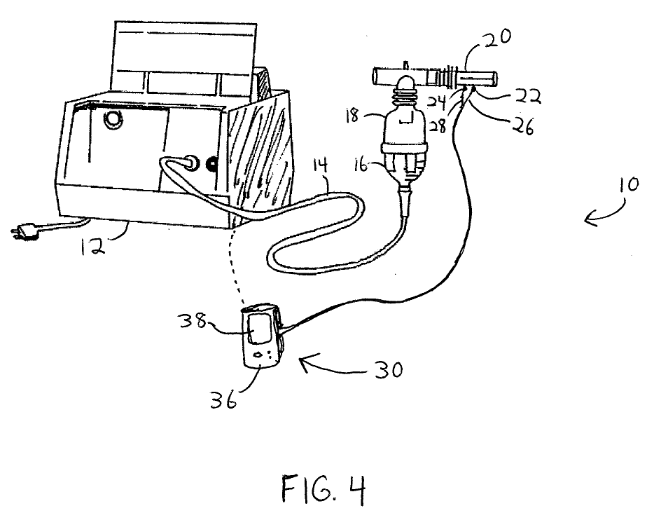

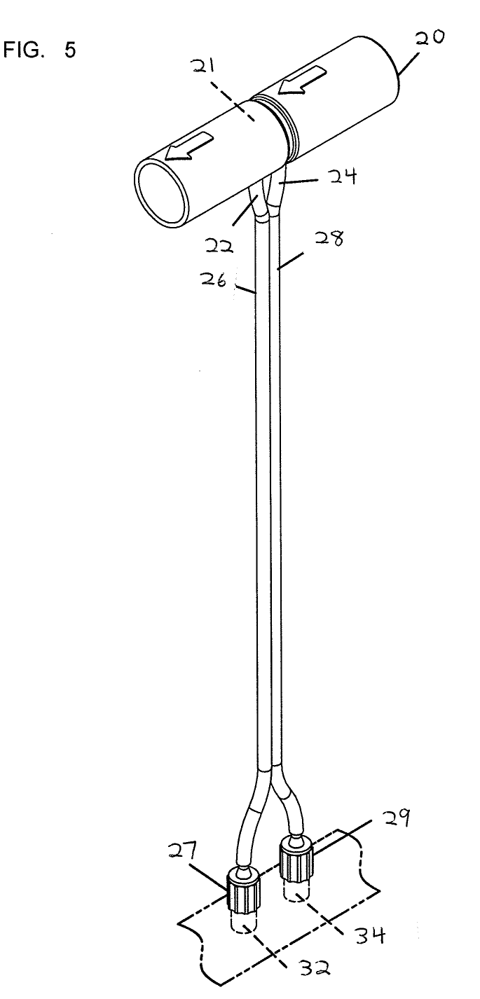

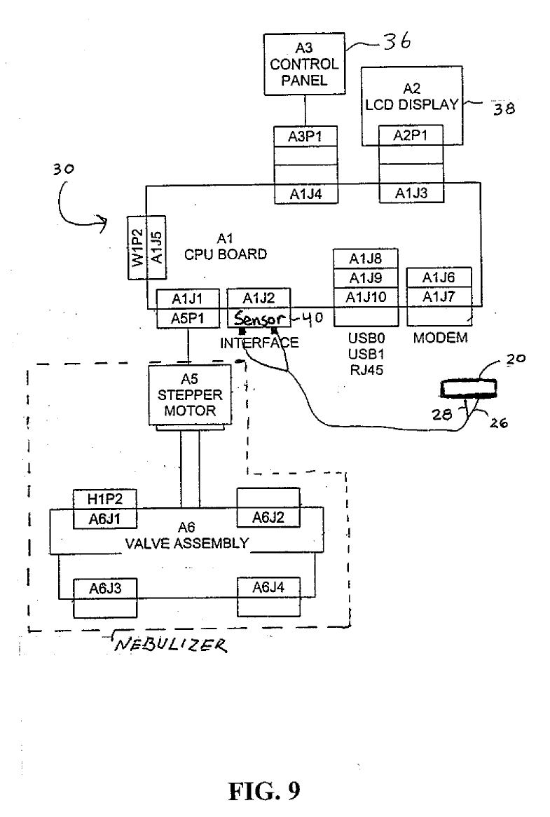

FIG. 4 depicts an embodiment of an aerosolized drug delivery system 10 in accordance with the present invention. System 10 includes an aerosol drug generator, a mouthpiece 20, and a handheld device 30. In the embodiment shown in FIG. 4, the aerosol drug generator is a nebulizer including a compressor 12 which delivers medication through tubing 14 to a cup 16 and dome 18. The medication is aerosolized in cup 16 and dome 18. The aerosolized medication then enters the mouthpiece 20. Mouthpiece 20 is adapted to be inserted into or to cover a patient's mouth during inhalation. Mouthpiece 20 includes a pair of sensing ports 22, 24. In the embodiment shown in FIG. 4, the sensing ports 22, 24 are air ports. The air ports 22, 24 are connected to handheld device 30 via a pair of flexible tubes 26, 28. Tubes 26, 28 engage the air ports 22, 24 of the mouthpiece 20 at one end and are coupled to air ports 32, 34 of the handheld device 30 via threaded couplings 27, 29 at the other end (as shown in FIG. 5). Handheld device 30 includes a differential air pressure sensor 40 (as shown in FIG. 9) in communication with the controller of handheld device 30. Alternatively, tubes 26, 28 can be attached to an intermediate sensor (not shown) for converting air pressure into an analog or digital signal which can be communicated to handheld device 30. Handheld device 30 includes a control panel 36 and a display component. In the embodiment shown in FIG. 4, the display component is an LCD panel display 38.

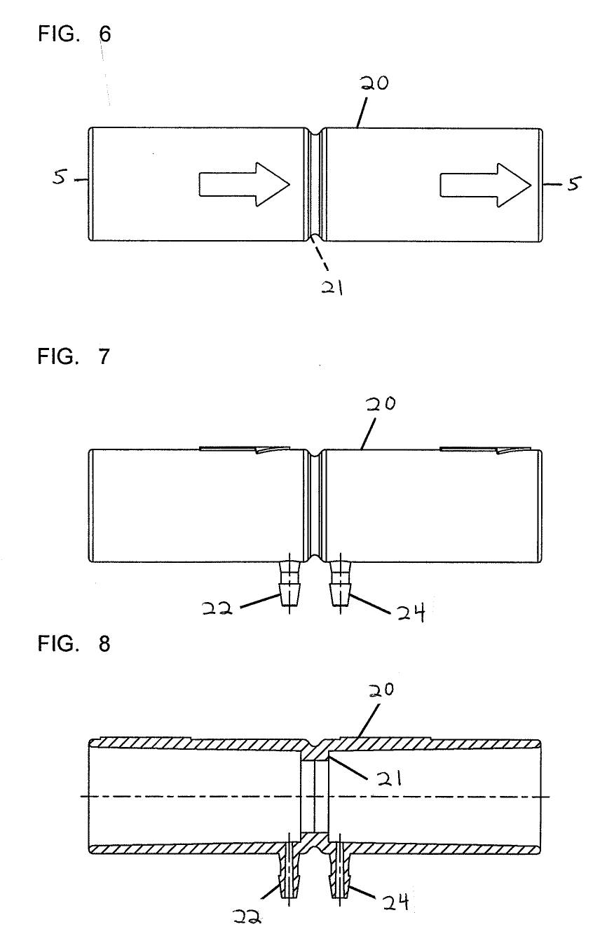

FIGS. 5-8 illustrate various views of mouthpiece 20 adapted for use with an aerosolized drug delivery system 10. Mouthpiece 20 is a mouthpiece through which a patient inhales. In some embodiments, a patient may also exhale through mouthpiece 20. Mouthpiece 20 defines an open ended tube having an interior flow restriction 21 and a pair of air ports 22, 24. The mouthpiece 20 may be generally cylindrical in form, as shown, or may assume alternative shapes. The flow restriction 21 may be a ring form, as shown, or may assume alternative configurations. The flow restriction 21 may be generally centered along the length of the mouthpiece tube or may be offset relative to center. It is envisioned that a variety of different mouthpiece configurations could be utilized in alternative designs suitable for use within system 10. Mouthpiece 20 may include a two-way airflow valve. Mouthpiece 20 is also discussed in U.S. Pat. No. 12,482,219 of Hansen et al., filed Jun. 10, 2009, the disclosure of which application being hereby incorporated by reference herein in its entirety.

FIG. 9 illustrates a somewhat diagrammatical schematic of aerosolized drug delivery system 10. As shown in FIG. 9, mouthpiece 20 is connected to a nebulizer. Flexible tubes 26, 28, connect the mouthpiece 20 to handheld device 30 via sensor 40. Sensor 40 is a pressure sensor incorporated into handheld device 30. Sensor 40 converts the pressure signal from tubes 26, 28 into an electrical signal.

Handheld device 30 includes sensor 40, control panel 36, and a display component. In the embodiment depicted in the figures, the display component is LCD panel display 38. Handheld device 30 also includes a signal receiving component, a data processing component, and a measurement component. The signal receiving component receives the electrical signal from sensor 40 that is derived from the pressure signal from tubes 26, 28. The data processing component is adapted to calculate an inspired flow rate based on the electrical signal received by the signal receiving component. A plurality of inspired flow rates may be calculated during a therapy session in which aerosolized drug is delivered to a patient. The inspired flow rate data may be stored in a storage component of the handheld device.

The data processing component is also adapted to calculate the amount of the aerosolized drug delivered to a patient based on the inspired flow rate, the amount of inhalation time, and a drug delivery coefficient. The inhalation time is the amount of time that the aerosolized drug is inhaled at the inspired flow rate. This inhalation time is measured by the measurement component of the handheld device 30.

LCD panel display 38 may display the inspired flow rate calculated by the data processing component. It may also display a desired flow rate, to allow a patient to compare his or her inspired flow rate to the desired flow rate, and to adjust his or her inspired flow rate accordingly. The data processing component may also quantitatively compare the inspired flow rate to a desired flow rate.

The data processing component may also calculate the percentage of time during which the aerosolized drug is inhaled in relation to the total time of a therapy session. The calculated percentage of time may be displayed on the LCD panel display 38.

In some embodiments, the display component of handheld device 30 includes a plurality of indicator lights. Each of these indicator lights corresponds to at least one inspired flow rate. Therefore, at a high inspired flow rate, one of the plurality of indicator lights would be activated, while at a low inspired flow rate, another of the plurality of indicator lights would be activated. In this manner, inspired flow rate data could be communicated to a patient using indicator lights. For example, embodiments of the present invention may include a user interface that consists of two buttons labeled start and stop, a liquid crystal display or other appropriate display, and eight different LED lights that represent eight storage areas for the data associated with eight different flow rates. Alternatively, LED lights may be arranged such that the lights create a bar graph, with each column representing an inspired flow rate, and the length of each column representing the amount of time spent at each inspired flow rate.

A description of the operation of an embodiment of the present invention follows. First, a user turns a power control switch to the ON position to initialize the trainer. A screen on the handheld device instructs the patient to load the nebulizer with the prescribed drug. The aerosol generator is activated by pressing the start button. Pressing the start button also activates the display component of the handheld device, and begins the collection of the timed air flow data. When the display component includes LED lights, the LED lights may be activated during inhalation only, serving as a visual trainer for the patient to observe during therapy. The display component may teach the patient the proper inhalation flow rates for optimum deposition of the aerosol in the lungs, for maximum therapeutic effect. When the therapy session is finished the stop button is pressed. The total amount of aerosol delivered to the patient for that therapy session is displayed by the display component, as a value in mg. Pressing the start button again causes compliance data for that session to be displayed. Specifically, the percentage of time of the total therapy session that the patient was inhaling the aerosol is displayed. The percentage of time may be further broken down into the percentage of time spent at each of various different flow rates, such as three different flow rates. This feature permits the care-giver to not only monitor the overall compliance of the patient, but also to monitor breathing compliance techniques necessary to achieve maximum drug effect. Pressing the start button again will return the display to the first screen displaying the total amount of aerosol delivered. To start a new session, the stop button may be pressed after the compliance display. The user may then follow the display instructions to fill the nebulizer and start the trainer and aerosol therapy session as before.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A system for delivering an aerosolized drug to a patient comprising:
an aerosol drug generator coupled to a mouthpiece including two sensing ports;
a pressure sensor connected to the two sensing ports;
a data processing component adapted to calculate an inspired flow rate based on a signal from the pressure sensor, and
a measurement component adapted to measure an amount of inhalation time during which an aerosolized drug is inhaled at the inspired flow rate,
wherein the data processing component is adapted to calculate an amount of the aerosolized drug delivered to the patient based on the inspired flow rate, the amount of inhalation time, and a drug delivery coefficient.

2. The system of claim 1, further comprising a display component adapted to display the inspired flow rate.

3. The system of claim 2, wherein the display component is further adapted to display a desired flow rate.

4. The system of claim 1, wherein the data processing component is further adapted to compare the inspired flow rate to a desired flow rate.

5. The system of claim 1, wherein the data processing component is further adapted to calculate a percentage of a time during which the aerosolized drug was inhaled in relation to a total time of a therapy session.

6. The system of claim 5, wherein the display component is further adapted to display the percentage of time.

7. The system of claim 1, wherein the data processing component is adapted to calculate a plurality of inspired flow rates.

8. The system of claim 1, wherein the mouthpiece comprises a two-way airflow valve.

9. A system for delivering an aerosolized drug to a patient comprising:
an aerosol drug generator coupled to a mouthpiece including two sensing ports;
a pressure sensor connected to the two sensing ports; and
a signal receiving component adapted to receive signals from the pressure sensor, and a data processing component adapted to calculate a plurality of inspired flow rates based on the signals from the pressure sensor.
a display component comprising a plurality of indicator lights, wherein each of the plurality of indicator lights corresponds to an inspired flow rate of the plurality of inspired flow rates.

10. The system of claim 9, wherein system further comprises a display component comprising a plurality of indicator lights, wherein each of the plurality of indicator lights corresponds to an inspired flow rate of the plurality of inspired flow rates.

11. The system of claim 9, wherein the computer system further comprises a measurement component adapted to measure an amount of inhalation time during which the aerosolized drug is inhaled at each of the plurality of inspired flow rates.

12. The system of claim 11, wherein the data processing component is further adapted to calculate an amount of the aerosolized drug delivered to the patient based the plurality of inspired flow rates, the amount of inhalation time, and a drug delivery coefficient.

13. The system of claim 9, wherein the data processing component is further adapted to calculate a percentage of a time during which the aerosolized drug was inhaled in relation to a total time of a therapy session.

14. The system of claim 13, wherein the display component is further adapted to display the percentage of time.

15. The system of claim 9, further comprising a valve controlled in response to a predetermined pressure sensor signal, said valve controlling an air flow through said aerosol drug generator during patient expiration.

16. A method of estimating an amount of aerosolized drug delivered to a patient during a therapy session using an aerosol generator, said method comprising:
calculating an inspired flow rate of the patient as the patient inhales an aerosolized drug through a mouthpiece by sensing a pressure differential across the mouthpiece;
measuring an amount of inhalation time during which the aerosolized drug is inhaled at the inspired flow rate; and
calculating the amount of aerosolized drug delivered to the patient based on the inspired flow rate, the amount of inhalation time, and a drug delivery coefficient.

17. The method of claim 16, further comprising displaying the inspired flow rate.

18. The method of claim 17, further comprising displaying a desired flow rate.

19. The method of claim 16, further comprising determining a period of patient expiration and controlling an air flow through said aerosol generator during said period.

20. The method of claim 15, further comprising calculating a percentage of a time during which the aerosolized drug was inhaled in relation to a total time of the therapy session.
